(19) United States
(12) Patent Application Publication
(10) Pub. No.: US 2012/0190586 A1
(43) Pub. Date: Jul. 26, 2012

(54) Synthetic Receptors for Identification of Protein Posttranslation Modifications

(76) Inventors: Marcey L. Waters, Carrboro, NC (US); Lindsey Ingerman James, Chapel Hill, NC (US)

(21) Appl. No.: 13/496,825

(22) PCT Filed: Oct. 13, 2010
(86) PCT No.: PCT/US10/52418

§ 371 Date: Apr. 10, 2012

(30) Foreign Application Priority Data
Oct. 15, 2009 (US) 61251983

(51) Int. Cl. C40B 50/04; G01N 33/68; C07K 1/32; C07D 341/00

(52) U.S. Cl. 506/25; 549/11; 436/86; 530/327

(57) ABSTRACT

A method of making a synthetic organic receptor that specifically binds to a modified amino acid is carried out by: reacting a plurality of monomers in a dynamic combinatorial library in the presence of a protein or peptide to form a reaction product, the protein or peptide comprising a modified nucleic acid, with the reacting step carried out under conditions in which at least one oligomer that specifically binds to the modified amino acid is selectively amplified in the reaction product; and then isolating or identifying from the reaction product at least one oligomer that specifically binds to the modified amino acid.

### RELATED APPLICATIONS

This application claims the benefit of under 35 U.S.C. §119(e) of U.S. Provisional Patent Application Ser. No. 61/251,983, filed Oct. 15, 2009 (Docket No. 5470-542Pr); the disclosure of which is incorporated herein by reference in its entirety.

### GOVERNMENT FUNDING

This invention was made with Government Support under Grant No. W911NF06-1-0169 from the Army Research Office and the Defense Threat Reduction Agency. The US Government has certain rights to this invention.

### BACKGROUND OF THE INVENTION

Protein post-translational modifications (PTMs) are used as signaling mechanisms in the cell, and play a role in many disease states, including cancer. Thus, significant effort is now focused on identifying which proteins are modified and under what conditions; the sites of modification in proteins; the enzymes involved; and the proteins that recognize and “read” the PTMs.

Two primary methods are currently used for identifying PTMs in: mass spectrometry (MS) and immunoassays. MS is a powerful tool for sequencing proteins, but is labor-intensive and time-consuming. Antibodies have been used extensively in Western blots and microarrays to determine methylation states in histones, and can be used on mixtures of proteins, but these have their own set of limitations. They are sequence-specific, such that new PTM sites or new proteins with PTMs cannot be identified in this way. For applications involving the recognition of PTMs in histone proteins, antibodies are limited because neighboring PTMs can influence the affinity of the antibody for its target sequence. In addition, antibodies cannot provide information regarding which PTMs occur within the same protein. Furthermore, antibodies cannot be used in intracellular assays. Lastly, recent literature has suggested that reproducibility with antibodies is often low.

### SUMMARY OF THE INVENTION

A first aspect of the invention is a method of making a synthetic organic receptor that specifically binds to a modified amino acid, comprising: reacting (e.g. in an aqueous media) a plurality of monomers in a dynamic combinatorial library in the presence of a protein or peptide to form a reaction product, the protein or peptide comprising a modified amino acid, with the reacting step carried out under conditions in which at least one oligomer that specifically binds to the modified amino acid is selectively amplified in the reaction product; and then isolating or identifying from the reaction product at least one oligomer that specifically binds to the modified amino acid.

A second aspect of the invention is a synthetic organic receptor that specifically binds to a modified amino acid, for use in detecting a protein or peptide comprising the modified amino acid; wherein the receptor is preferably (a) an oligomer reaction product of a dynamic combinatorial library of monomers, or (b) an analog thereof.

A further aspect of the present invention is a method of collecting or detecting a protein or peptide (e.g., as described herein) comprising a modified amino acid, method comprising, contacting the protein or peptide to a synthetic organic receptor as described herein, e.g., in an aqueous media.

In some embodiments of the foregoing, the modified amino acid is selected from the group consisting of monomethyl lysine, dimethyl lysine, trimethyl lysine, acetyl lysine, monomethyl arginine, symmetric dimethyl arginine, asymmetric dimethyl arginine, citrulline, phosphoserine, phosphothreonine, phosphotyrosine, 3-nitrotyrosine, oxomethionine, S-methylmethionine, S-adenosylmethionine, and glycosyl amino acids.

In some embodiments, the glycosyl amino acid is selected from the group consisting of glycosylated serine, glycosyl threonine, glycosyl tyrosine, and glycosyl asparigine.

In some embodiments, the modified amino acid is a glycosylated amino acid comprising a glycosyl selected from the group consisting of glucose, galactose, mannose, fucose, GalNAc, GIcNAc and NANA.

In some embodiments, each of the monomers is a compound of the formula A-B-C, wherein:

A is a first reactive group;

B is a linking group; and

C is a second reactive group that reversibly covalently bonds to the first reactive group.

In some embodiments, the first and second reactive groups are each independently selected from the group consisting of amino groups, aldehyde groups, keto groups, thiol groups, thioester groups, olefinic groups, alcohol groups, carbonyl groups, hydrazine groups, hydroxylamine groups and borate groups.

In some embodiments, each of the linking groups is independently selected from the group consisting of alkyl, aryl, alkylaryl, alkylarylalkyl, amide, oligoamide, ester, urea, guanidinium, and ether linking groups, and combinations thereof.

In some embodiments, the oligomer is a linear or cyclic, preferably cyclic, molecule consisting of from 2 or 3 to 15, 20 or 30 monomers covalently coupled to one another. In some embodiments, the oligomer are coupled to one another by disulfide, imine, acyl-hydrazone, amide, acetal, ester, or thioester linkages.

In some embodiments, the receptor is coupled to a detectable group or solid support.

The present invention is explained in greater detail in the drawings herein and the specification set forth below. The disclosures of all United States Patent references cited herein are to be incorporated by reference herein in their entirety.

### BRIEF DESCRIPTION OF THE DRAWINGS

FIG. 1. Dynamic combinatorial library.

FIG. 2. Semi-preparative HPLC trace of an A₂B biased library.

FIG. 3. Analytical LC traces of purified rac-A₂B (top) and meso-A₂B (bottom).

FIG. 4. Mass spectra of purified rac-A₂B and meso-A₂B.

FIG. 5. Fluorescence anisotropy of rac-A₂B with H3 KMe₃ (10 mM sodium phosphate buffer pH 8.5).

FIG. 6. Fluorescence anisotropy of rac-A₂B with H3 KMe₂ (10 mM sodium phosphate buffer pH 8.5).

FIG. 7. Fluorescence anisotropy of rac-A₂B with H3 KMe (10 mM sodium phosphate buffer pH 8.5).

FIG. 8. Fluorescence anisotropy of rac-A₂B with H3 K (10 mM sodium phosphate buffer pH 8.5).

FIG. 9. Fluorescence anisotropy of meso-A₂B with H3 KMe₃ (10 mM sodium phosphate buffer pH 8.5).

FIG. 10. Fluorescence anisotropy of meso-A₂B with H3 KMe₂ (10 mM sodium phosphate buffer pH 8.5).

FIG. 11. Fluorescence anisotropy of rac-A₂B with H3 KMe₃ where Arg8 has been mutated to Gly8 (10 mM sodium phosphate buffer pH 8.5).

FIG. 12. Histone 3 peptide (WGGG-GKMe₃) used as the titrant in ITC experiments to confirm A₂B binding to KMe₃.

FIG. 13. ITC binding curve from the titration of WGGG-GKMe₃ into rac-A₂B, giving a K_{d} of 20.0 μM.

FIG. 14. ITC binding curve from the titration of WGGG-GKMe₃ into meso-A₂B, giving a K_{d} of 12.8

### DETAILED DESCRIPTION OF EMBODIMENTS OF THE INVENTION

“Protein” and “peptide” as used herein may be any natural or synthetic protein or peptide, of any suitable length, e.g., from 2 or 5 to 100, 200 or 500 amino acids, or more. The protein or peptide preferably comprises, consists of or consists essentially of a modified amino acid (e.g., preferably one or more units of a single type of modified amino acid).

“Modified amino acid” as used herein may be any natural (e.g. post-translationally modified) or synthetic modified amino acid, including but not limited to monomethyl lysine, dimethyl lysine, trimethyl lysine, acetyl lysine, monomethyl arginine, symmetric dimethyl arginine, asymmetric dimethyl arginine, citrulline, phosphoserine, phosphothreonine, phosphotyrosine, 3-nitrotyrosine, oxomethionine, S-methylmethionine, S-adenosylmethionine, glycosylated serine, glycosyl threonine, glycosyl tyrosine, glycosyl aspargine, and other glycosyl amino acids, where glycosyl refers to any saccharide or oligosaccharide.

“Glycosyl” or “glycosyl group” as used herein may be any suitable glycosyl group (e.g., and N- or O-linked glycosyl group), including but not limited to glucose, galactose, mannose, fucose, GalNAc, GIcNAc, and oligosaccharides (including but not limited to lactose, sialyl Lewis X, etc.) (see, e.g., U.S. Pat. No. 7,332,355; see also U.S. Pat. No. 7,276,475).

“Linking group” as used herein refers to any suitable group having two covalent bonds, one to each linked group. Example linking groups include but are not limited to, those comprising, consisting of or consisting essentially of alkyl, aryl, alkylaryl, or alkylarylalkyl linking groups (each of which may optionally contain 1 or more, e.g., 2, 3, or 4 or more hetero atoms such as N, O, or S, and each of which is unsubstituted or optionally substituted with one or more (e.g., 1, 2, 3, 4) additional functional groups) and combinations thereof. Linking group may comprise include amides, oligoamides, ethers, esters, etc, and combinations thereof. Thus, and stated differently, example linking groups include, but are not limited to, those comprising alkylenes, optionally-substituted aryls, and optionally-substituted heteroaryls, and combinations thereof, wherein the linking group may be terminated or interrupted by one or more oxygen atoms, sulphur atoms, keto groups, —O—CO— groups, —CO—O— groups, or —NR groups in which R is an alkyl or aryl group. See, e.g., U.S. Pat. No. 7,595,292. Numerous additional examples of linking groups are known which can be used to carry out the present invention. See, e.g., U.S. Pat. Nos. 7,601,851; 7,601,691; 7,598,290; 7,598,234; and 7,595,345, the disclosures of which are incorporated by reference herein in their entirety. The linking group may optionally have one or more additional functional groups covalently coupled or substituted therein, including amines and alkylamines, hydroxyl groups, carboxyl groups, esters, ketones, amide groups, ammonium groups, ethers, sulfates, sulfonates, phosphates, phosphonates, or any other functional group that may contribute to binding and/or water solubility (see also “functional group” below).

“Alkyl,” as used herein, refers to a straight, branched chain, or cyclic hydrocarbon, for example containing from 1 to 10 or 20 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, 3-methylhexyl, 2,2-dimethylpentyl, 2,3-dimethylpentyl, and the like, which alkyl may be substituted or unsubstituted. The alkyl may be saturated or unsaturated (e.g., contain one or more double or triple bonds, also referred to as an alkenyl or alkynyl group). The alkyl may be unsubstituted or substituted one or more (e.g., 2, 3, 4, 5) times, e.g., with independently selected reactive groups and/or functional groups as described herein. When used as a linking group alkyl as described herein includes two covalent bonds, one to each linked group (A, C).

“Aryl,” as used herein, refers to a monocyclic carbocyclic ring system or a bicyclic carbocyclic fused ring system having one or more aromatic rings. Representative examples of aryl include azulenyl, indanyl, indenyl, naphthyl, phenyl, tetrahydronaphthyl, and the like, which aryl may be substituted or unsubstituted. The aryl may be unsubstituted or substituted one or more (e.g., 2, 3, 4, 5) times, e.g., with independently selected reactive groups and/or functional groups as described herein. When used as a linking group aryl as described herein includes two covalent bonds, one to each linked group (A, C). Aryl as used herein includes heteroaryl, unless specified otherwise.

“Alkylaryl” as used herein refers to an alkyl as described above covalently coupled to an aryl as described above.

“Alkylarylalkyl” as used herein refers to an alkyl as described above covalently coupled to an aryl as described above, the aryl in turn coupled to an additional alkyl as described above.

“Heteroaryl” as used herein means a cyclic, aromatic hydrocarbon in which one or more carbon atoms have been replaced with heteroatoms. If the heteroaryl group contains more than one heteroatom, the heteroatoms may be the same or different. Examples of heteroaryl groups include pyridyl, pyrimidinyl, imidazolyl, thienyl, furyl, pyrazinyl, pyrrolyl, pyranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, naphthyridinyl, quinoxalinyl, isothiazolyl, and benzo[b]thienyl. Preferred heteroaryl groups are five and six membered rings and contain from one to three heteroatoms independently selected from O, N, and S. The heteroaryl may be unsubstituted or substituted one or more (e.g., 2, 3, 4, 5) times, e.g., with independently selected reactive groups and/or functional groups as described herein.

“Functional group” as used herein means any polar, nonpolar, hydrophilic, lipophilic, or charged unit or subunit, or electron donor or electron acceptor group. Examples include but are not limited to simple functionalities like amino and imino groups and derivatives thereof, hydroxy and mercapto groups and derivatives thereof, oxo and thioxo groups, formyl and thioformyl groups, aryl groups, substituted aryl groups; phenyl groups, substituted phenyl groups, pyridyl groups and derivatives thereof, carboxy groups and carboxylato groups and derivatives thereof, alkyloxycarbonyl groups, (di)thiocarboxy groups and derivatives thereof, (di)thiocarboxylato groups, carbamoyl groups and derivatives thereof, sulfo, sulfino and sulfeno groups and derivatives thereof, alkyloxysulfonyl, alkyloxysulfinyl and alkyloxysulfenyl groups, sulfamoyl, sulfinamoyl and sulfenamoyl groups and derivatives thereof, cyano and (iso)(thio)cyanato groups, hydroperoxy groups, nitroso groups, hydroxyamino groups, hydrazino groups, —NR₁R₂, —⁺NHR₁R₂ and —⁺NR_{I}R₂R₃ groups, wherein R₁, R₂, and R₃ are identical or different and represent alkyl, cycloalkyl, alkylcycloalkyl, aryl, alkylaryl with 1 to 40 C atoms, —⁺OR₁R₂ groups wherein R₁ and R₂ are identical or different and represent alkyl, cycloalkyl, alkylcycloalkyl, aryl, alkylaryl with 1 to 40 C atoms, hydrazide groups and any other suitable groups known to a person skilled in the art. See, e.g., J.-M. Lehn et al., US Patent Application No. 2004/0029172 (published Feb. 12, 2004).

“Reactive group” as used herein, typically with reference to first and second reactive group, may be any functional group capable of reversible covalent reaction with another reactive group on another monomer in a dynamic combinatorial library. Examples for functional groups which may react with other functional groups under reversible bond formation include amino groups, aldehyde groups, keto groups, thiol groups, olefinic groups, alcohol groups, carbonyl groups, hydrazine groups, hydroxylamine groups and borate groups. Examples of reversible covalent reactions with the above-mentioned groups involved are those where carbonyl groups react under the formation of imines, acyl-hydrazones, amides, acetals, thioesters, and esters. In particular, the reaction of amino groups with carbonyl groups to imines, oximes or hydrazones is useful. Reactions such as thiol exchange in disulphides or alcohol exchange in borate esters are further examples, as well as reversible Diels-Alder and other thermal- or photoinduced rearrangements like sigmatropic and electrocyclic rearrangements, and Michael reactions or alkene metathesis using catalysts that may be soluble in water. Photoinduced interconversions represent another possibility leading to photodynamic combinatorial processes. See, e.g., J.-M. Lehn et al., US Patent Application No. 2004/0029172 (published Feb. 12, 2004).

“Detectable group” as used herein include, but is not limited to, radiolabels (e.g., ³⁵S, ¹²⁵I, ³²P, ¹³H, ¹⁴C, ¹³¹I), enzyme labels (e.g., horseradish peroxidase, alkaline phosphatase), gold beads, chemiluminescence labels, ligands (e.g., biotin, antibodies), peptides (e.g. myc peptide, flagtag) and/or fluorescence labels (e.g., fluorescein). Such detectable groups may be coupled to the parent molecule or receptor in accordance with known techniques

Dynamic combinatorial libraries. Dynamic combinatorial libraries can be prepared in accordance with known techniques or variations thereof that will be apparent to those skilled in the art in view of the instant disclosure. See, e.g., J.-M. Lehn et al., US Patent Application No. 2004/0029172 (published Feb. 12, 2004), see also L. Ingerman and M. Waters, J. Org. Chem. 74, 111-117 (2009); and Corbet, P. T.; Leclaire, J.; Vial, L.; West, K. R.; Wietor, J. L.; Sanders, J. K. M.; Otto, S. Chem. Rev. 106, 3652-3711 (2006).

Applications and uses. This method provides a new, facile route for generation of selective small-molecule receptors for any protein PTM. Such receptors can be synthetically modified to attach a dye molecule in a straight-forward manner to convert the receptor into a sensor. These small molecule receptors and sensors could be used in any application that currently uses an antibody. Small molecule receptors have a number of advantages over antibodies, including:
Small molecule receptors for PTMs can function as replacements for antibodies in Western blots, microarrays, and any other assay that currently uses antibodies.
Small molecule receptors represent a single chemical species, such that the results from assays such as Western blots, microarrays should be highly reproducible.
Small molecule receptors may be applicable to intracellular assays which are not possible with antibodies.
The small molecule receptors can be developed to be non-sequence selective (i.e., will bind to the PTM independent of the surrounding peptide sequence), so that the receptors can be used as a general sensor the same PTM in any protein or sequence. This would allow for a rapid method for identifying the PTM in a previously unknown protein.
Development of sequence-selective receptors is also possible, which could directly replace antibodies that are currently available.

The present invention is explained in greater detail in the following non-limiting examples.

### Experimental

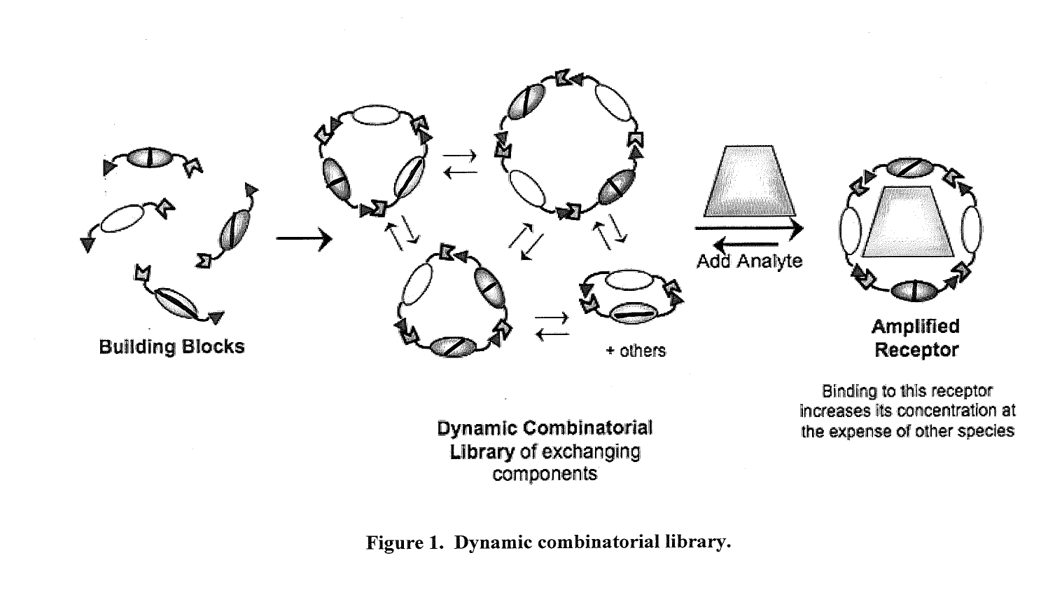

To date, small molecules receptors are not commonly utilized as sensors in biological assays because of the difficulty in identifying high affinity, high selectivity small molecule receptors. Dynamic combinatorial chemistry (DCC) provides a high-throughput method for developing small-molecule receptors for protein post-translational modifications (PTMs), making it feasible to use small molecule receptors in assays that typically use antibodies. DCC methodology utilizes the equilibration of species via reversible covalent bond formation to create a dynamic library of potential receptors (FIG. 1). When this thermodynamically controlled mixture is incubated with an analyte of interest (such as trimethyl lysine), the library responds by shifting the equilibrium towards the receptor(s) that best binds the analyte. This differs from a traditional (static) combinatorial library in that there is simultaneous generation of the library and amplification of the host(s). Thus, it allows for the synthesis of receptors that would be difficult to synthesize via traditional approaches, and results in host structures that may not be predicted a priori.

We have used disulfide exchange and thioester exchange as the equilibrating reaction. Receptors identified by disulfide exchange can be used directly in any in vitro assay. Receptors identified from thioester exchange must be resynthesized with amide or ester linkages in place of thioesters prior to application. Libraries of equilibrating compounds are analyzed by HPLC-MS. A comparison is made of a library without any of the target PTM and in the presence of the PTM. Species that are amplified in the presence of the PTM are identified as receptors for that PTM. PTMs are incorporated into short peptides for screening purposes, although whole proteins could also be used for library screening.

### EXAMPLES

Examples of a dynamic combinatorial library and receptors for PTMs identified by disulfide exchange are shown in Schemes 1-2 below. In the case of compound 1, all three possible stereoisomers were identified as receptors. For compound 2, only one symmetrical isomer was identified as a receptor, but whether it is 2a or 2b has yet to be determined. In the case of compound 3, we have not yet determined which isomer(s) are receptors for PTMs. Compound 1 is a receptor for trimethyllysine that shows significant selectivity over unmodified amino acids and other PTMs, including mono- and dimethyllysine, arginine, mono- and dimethyl arginine (both symmetric and asymmetric). Moreover, it binds to trimethyllysine with an affinity that is similar to that of the native protein receptor. This is a non-sequence selective receptor, so it can in theory recognize any trimethyllysine PTM in any protein.

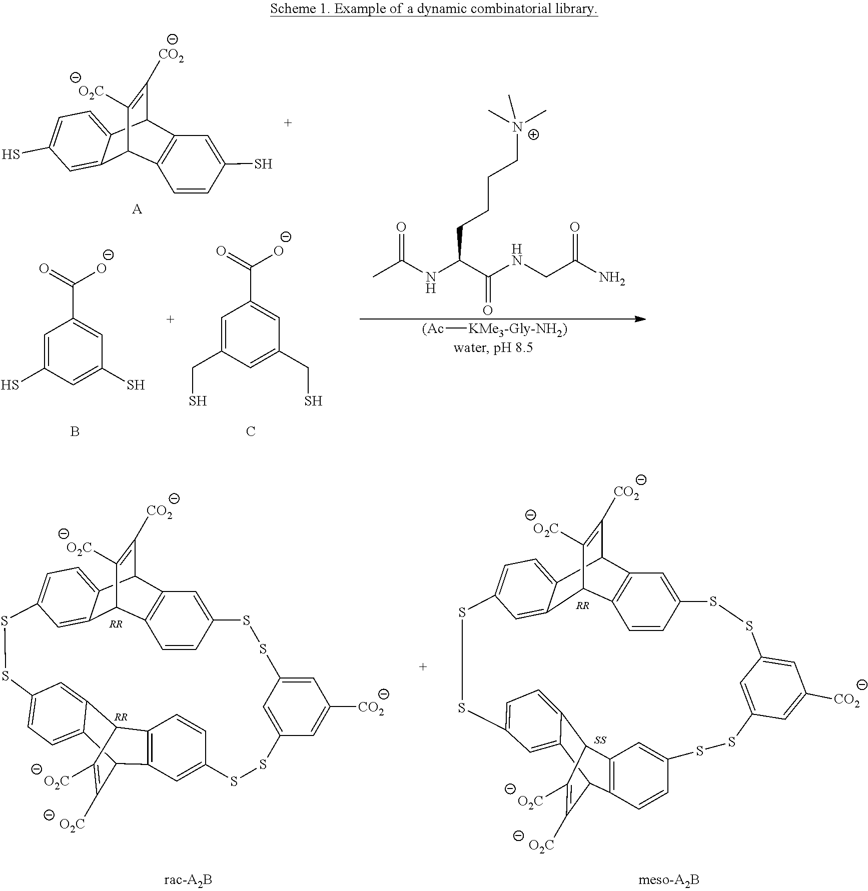

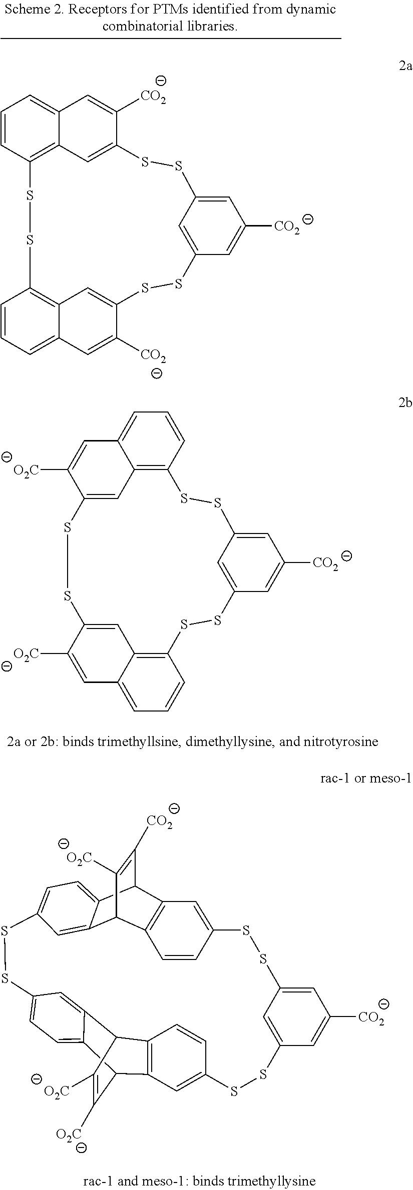

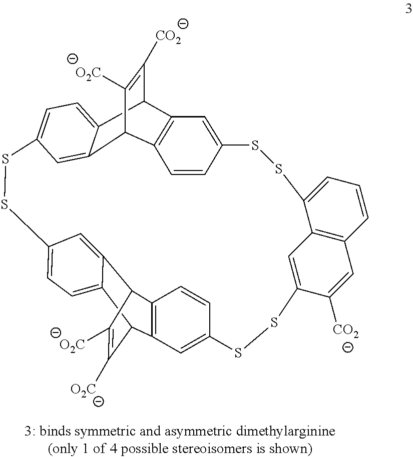

### Experimental

Understanding the role of post-translationally modified histone proteins in gene expression is crucial to the study of both development and disease (Bhaumik, S. R. et al., Nature Struct. Molec. Biol. 2007, 14, 1008-1016.; Wang, G. G. et al., Trends in Molec. Med. 2007, 13, 363-372.). Many post-translational modifications (PTMs) have been shown to function by recruiting non-histone proteins to chromatin, dictating the higher-order chromatin structure in which DNA is packaged, and resulting in gene expression or gene silencing depending on the type and location of the PTM (Jenuwein, T.; Allis, C. D. Science 2001, 293, 1074-1080; Kouzarides, T. Cell 2007, 128, 693-705). One such covalent modification is the site-specific methylation of Lys, which can be mono-, di-, or tri-methylated. Depending on the specific Lys residue that is modified as well as the degree of methylation, different proteins are recruited, resulting in variable transcriptional outcomes (Martin, C.; Zhang, Y. Nature Rev. Molec. Cell Biol. 2005, 6, 838-849).

There is much interest in mapping out the differences in Lys methylation between normal and disease states. Standard methods for identifying PTMs use antibodies, but these have some inherent limitations (Blow, N. Nature 2007, 447, 741-744; Kazanecki, C. C. et al., J. Cell. Biochem. 2007, 102, 925-935.). In contrast, the use of small molecule receptors for the recognition and identification of PTMs has remained largely unexplored (An aptamer that binds acetyl lysine was recently reported; see: Williams, B. A. R. et al., J. Am. Chem. Soc. 2009, 131, 6330-63310). While synthetic receptors have some significant potential advantages, such as the possibility of being used within cells, utilizing synthetic receptors for molecular recognition often presents the challenge of poor selectivity. Furthermore, synthetic modification for optimization is often difficult and labor-intensive.

Dynamic combinatorial chemistry (DCC) is an attractive alternative to the rational design of synthetic receptors in that it allows molecular recognition to guide the synthesis of complex host systems from simple building blocks (for reviews see: Corbett, P. T. et al., Chem. Rev. 2006, 106, 3652-3711; Lehn, J-M. Chem. Eur. J. 1999, 5, 2455-2463; Ladame, S. Org. Biomol. Chem. 2008, 6, 219-226; Ludlow, R. F.; Otto, S. Chem. Soc. Rev. 2008, 37, 101-108; Otto, S. Curr. Opin. Drug Disc. Devel. 2003, 6, 509-520). These building blocks are linked reversibly under thermodynamic control to produce an equilibrium mixture of potential receptors. In the presence of a molecular target, favorable host-guest binding interactions drive the synthesis and amplification of the best receptor(s) at the expense of other oligomers. We report here the use of DCC as a novel approach for the development of selective synthetic receptors for trimethyl lysine (KMe3) that bind with comparable affinities and selectivites to a native protein receptor, the HP1 chromodomain (Hughes, R. et al., Proc. Nat. Acad. Sci. USA 2007, 104, 11184-11188; Jacobs, S. A. et al., Science 2002, 295, 2080-2083; Nielsen, P. R. et al., Nature 2002, 416, 103-107).

The proteins that recognize trimethyl lysine (KMe3) have in common an aromatic pocket that binds KMe3 via cation-π and van der Waals interactions (Taverna, S. D. et al., Nat. Struct. Mol. Biol. 2007, 14, 1025-1040.).⁷′ Thus, we chose to explore building blocks that could form an aromatic cage for KMe3, inspired by Dougherty's cyclophane receptor (Ma, J. C.; Dougherty, D. A. Chem. Rev. 1997, 97, 1303-1324) and used previously by Sanders and Otto (Otto, S. et al., Science 2002, 297, 590-593; Corbett, P. T. et al., Chem. Eur. J. 2008, 14, 2153-2166). Building blocks A-C (Scheme 3) were synthesized, combining a rigid, curved binding surface in monomer A, aromatic groups to facilitate cation-π interactions, and carboxylates for water solubility. Disulfide exchange was selected as the reversible reaction for generation of dynamic combinatorial libraries (DCLs) as it is compatible with most protein functional groups in aqueous solution at close to neutral pH (Otto, S. et al., J. Am. Chem. Soc. 2000, 122, 12063-12064; Ramstrom, O.; Lehn, J.-M. ChemBioChem 2000, 1, 41-48). A DCL was prepared by mixing equimolar amounts of building blocks A-C (7.5 mM total) in water at pH 8.5. Upon reaching equilibrium, the resulting DCL was analyzed by LC-MS (data not shown). In the absence of a guest molecule, two major constituents are present, BC and ABC as well as numerous smaller peaks corresponding to other macrocyclic library members.

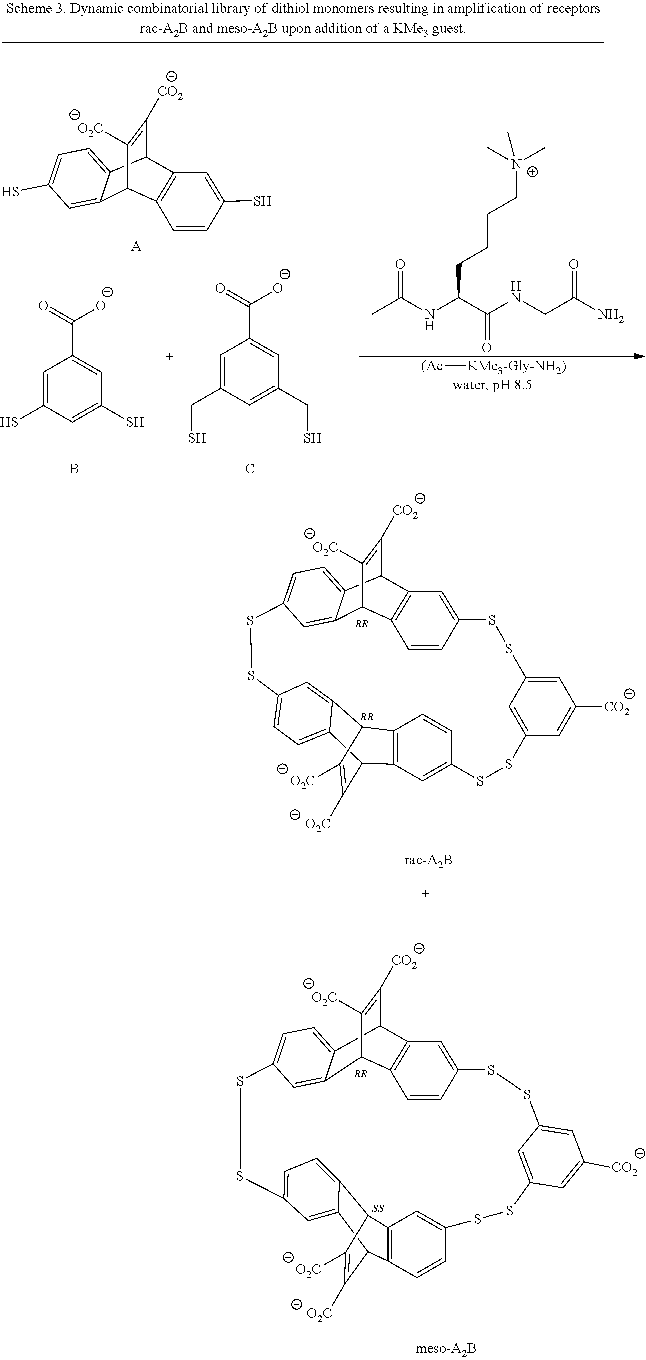

The introduction of the dipeptide Ac—KMe₃-G-NH₂ as a template resulted in significant changes in the composition of the library. Upon reaching equilibrium, the amplification of two peaks was observed, corresponding to the trimeric macrocycles rac- and meso-A₂B as identified by ESI-MS (data not shown). Roc-A₂B is the larger of the two peaks in both the templated and untemplated DCLs, consistent with previous reports and confirmed by NMR upon isolation.¹⁰ A similar degree of amplification was observed using an 8-residue histone tail sequence containing KMe3 as the guest, indicating that other nearby sidechains do not significantly influence binding.

Using a biased library in which building blocks A and B were mixed in a 2:1 ratio (7.5 mM total), the selectivity of the receptors for different methylation states of lysine was investigated, using Ac—KMeₙ-G-NH₂ as the guests (n=0-3). A diverse library was generated in the absence of a template, with A₂ and A₂B₃ as the dominant species and rac-A₂B and meso-A₂B constituting only 2.4% and 1.1% of the total library composition respectively. The amplification of both A₂B diastereomers was dependent on the extent of methylation, with approximately 10-fold amplification for KMe3 and less than 2-fold amplification for Lys (data not shown), suggesting significant selectivity for the different methylation states.

Both A₂β isomers were isolated for further studies using semi-preparative HPLC. NMR studies of the KMe3 dipeptide in the presence of rac-A₂B exhibit upfield shifting of the lysine methyl groups by 0.83 ppm at 25° C., confirming that the binding event is indeed occurring at the site of modification (No NMR studies were performed with meso-A₂B because of limited availability of this compound due to significant co-elution with rac-A₂B.).

Fluorescence anisotropy was used to measure the dissociation constant of rac- and meso-A₂B to a peptide consisting of residues 5-11 of histone 3 (H3 K9Meₙ), with each of the methylation states at Lys 9 as well as an N-terminal carboxyfluorescein (FAM) for fluorescence detection. Binding affinities for H3 K9Me3 were also confirmed by ITC (see below).

H3 K9Meₙ: FAM-QTAR-KMeₙ-STG-NH₂, n=0-3

The H3 K9Me3 peptide was found to bind both rac- and meso-A₂B with low micromolar affinity, with greater than 2-fold selectivity over its dimethylated counterpart (Table 1). The mono- and umethylated peptides exhibited considerably weaker affinity to rac-A₂B, with no appreciable binding to the unmethylated histone tail. Moreover, comparison to native HP1 chromodomain indicates that the affinities of rac- and meso-A₂B to H3 K9Me3 are similar to the native protein, while demonstrating better selectivity over H3 K9Me2, and weaker absolute binding affinity to both H3 K9Me and H3 K9.

TABLE 1
Dissociation constants for H3 histone tail peptides with varying
methylation states at K9 as determined by fluorescence anisotropy.^{a}

| | rac-A₂B | meso-A₂B | HP1 chromodomain |
|---|---|---|---|
| peptide | K_{d} (μM)^{b} | K_{d} (μM)^{b} | K_{d} (μM)^{e} |
| H3 K9Me₃ | 25 (3)^{c} | 28 (4)^{c} | 10 |
| H3 K9Me₂ | 58 (10) | 73 (9) | 15 |
| H3 K9Me | 166 (50) | N.A.^{d} | 96 |
| H3 K9 | >1200 | N.A.^{d} | >1000 |
| H3 K9Me₃ | 34 (8) | N.A.^{d} | — |
| R8G | | | |

^{a}Binding assays were performed at 27° C. in 10 mM phosphate buffer (pH 8.5).
^{b}Errors are shown in parentheses.
^{c}Values for rac- and meso-A₂B binding to H3 K9Me3 as measured by ITC are 20 μM and 13 μM, respectively.
^{d}Not available due to limited material (meso-A₂B), although binding is expected to be weak and comparable to that of rac-A₂B.
^{e}Values are taken from reference 6a. Binding assays were performed at 15° C. in pH 7.5 phosphate buffer.

A mutant H3 tail with Gly in place of Arg8 exhibited comparable affinity to that of the native sequence (Table 1), indicating that the adjacent basic Arg does not contribute significantly to binding. Additionally, Lys9 was mutated to Gly, which resulted in total loss of binding to rac-A₂B.

In conclusion, we report the identification of a small molecule receptor for KMe3 that exhibits both comparable affinity and selectivity to the native HP1 chromodomain. The observed selectivity for higher methylation states is likely due to differences in size and desolvation penalty. The comparable binding affinity to the native protein is impressive given that the synthetic receptor appears to bind only to the KMe3 sidechain, whereas the chromodomain also binds to the surrounding sequence. Such a sequence-independent receptor could be used to identify unknown sites of Lys methylation, unlike antibodies, which are sequence selective. To our knowledge this is the first example of a small molecule synthetic receptor that exhibits selective recognition of PTMs. This work validates DCC as a viable method to generate receptors that can distinguish between very slight differences in template structure, and suggests that DCC may be a promising method for developing affinity reagents for PTMs. Having gained insight into the features required for recognition, we are presently using DCC to optimize the binding affinity and selectivity for lysine PTMs.

Peptide Synthesis. All peptide synthesis was performed on a Tetras Peptide Synthesizer using Peptides International CLEAR-Amide resin. Peptides were synthesized on a 0.06 mmole scale. All amino acids with functionality were protected during synthesis. Coupling reagents were HOBt/HBTU in DMF. For the dipeptides, the N-terminus was acylated with a solution of 5% acetic anhydride and 6% 2,6-lutidine in DMF. Peptides synthesized for fluorescence anisotropy were capped with 2 equivalents of 5(6)-Carboxyfluorescein and coupled in the dark with standard coupling reagents overnight. Cleavage was performed by hand with a cocktail of 95% TFA/2.5% triisopropylsilane/2.5% H₂O for 3 hours. Peptides were purified by semipreparative reversed-phase HPLC on a C18 column at a flow rate of 4 mL/min. Peptides were purified with a linear gradient of A and B (A: 95% H₂O/5% CH₃CN with 0.1% TFA, B: 95% CH₃CN/5% H₂O with 0.1% TFA) and elution was monitored at 214 nm. Once purified, peptides were lyophilized to powder and characterized by ESI-MS.

Methylated peptides were synthesized with either 2 equivalents of Fmoc-Lys(Boc)(Me)-OH purchased from BaChem or Fmoc-Lys(Me)₂-OH.HCl purchase from Anaspec and coupled for 10-12 hours. The trimethyl lysine-containing peptides were synthesized by reacting the corresponding dimethylated peptides (0.6 mmol scale) prior to cleavage from the resin with MTBD (10.8 μL, 0.075 mmol) and methyl iodide (37.4 μL, 0.6 mmol) in DMF (5 mL) for 5 hours with bubbling N₂ in a peptide synthesis flask stoppered with a vented septum. After washing the resin with DMF (3×), CH₂Cl₂ (3×), and drying, the peptide was cleaved as normal.

Monomer Synthesis. Monomers A and B were synthesized following the reported procedure of Corbett, Sanders, and Otto (S. Chem. Eur. J. 2008, 14, 2153-2166).

Monomer C was synthesized following the reported procedure of Field and Kirrstetter Staab, H. A. (Kirrstetter, R. G. H. Liebigs Ann. Chem. 1979, 886).

Dynamic Combinatorial Chemistry. The relevant building blocks were individually dissolved in water, adding sufficient 1.0 M aqueous NaOH to fully deprotonate the thiols and carboxylic acids on the building blocks, using sonication when necessary. The pH of each solution was then adjusted to 8.5 using 1.0 M aqueous HCl and 1.0 M aqueous NaOH. In the unbiased A.B.C libraries, aliquots of each monomer solution were combined in a 2 mL LC-MS vial to reach a final concentration of 2.5 mM of each monomer. In the biased A₂B libraries, aliquots of each monomer solution were combined in a 2 mL LC-MS vial to reach a final concentration of 5 mM A and 2.5 mM of B. For the templated libraries, an aliquot of peptide guests dissolved in water were added to the reactions to reach a final concentration of 7.5 mM peptide. Any remaining volume was made up with water. The vials were capped and analyzed at various time points.

Analytical LC-MS. LC-MS was carried out on an Agilent Rapid Resolution LC-MSD system equipped with an online degasser, binary bump, autosampler, heated column compartment, and diode array detector. All separations were performed using 5 mM NH₄OAc H₂O-acetonitrile gradients at pH 5 and a Halo C18 column (4.6×100 mm, 2.7 micron). The MS was performed using a single quad mass spectrometer. Mass spectra (ESI+) were acquired in ultrascan mode by using a drying temperature of 350° C., a nebulizer pressure of 45 psi, a drying gas flow of 10 L/min, and a capillary voltage of 3000 V. The reactions were monitored weekly (3 μL injections) until equilibrium was reached after about 3 weeks. The chromatography of library A.B.C (data not shown) was carried out at 50° C. with gradient A (Table 2). The chromatography of library A₂B was carried out with gradient B (Table 2) using a gradient temperature, going from 50° C. to 40° C., left to right. The peak areas were integrated at 254 nm and the amplification factors were calculated (A.F.=% area of A₂B in templated DCL/% area of A₂B in untemplated DCL).

TABLE 2
Analytical LC methods use to analyze DCC
libraries A•B•C (Method A) and A₂B (Method B).

| | | |
|---|---|---|
| Time (min) | % B | Flow Rate (mL/min) |

Gradient A

| | | |
|---|---|---|
| 0.00 | 0.0 | 1.0 |
| 3.00 | 30.0 | 1.0 |
| 7.00 | 32.3 | 1.0 |
| 7.30 | 32.3 | 1.0 |
| 7.35 | 32.3 | 0.6 |
| 9.00 | 32.3 | 0.6 |
| 9.10 | 32.3 | 1.0 |
| 10.80 | 34.0 | 1.0 |
| 10.90 | 100.0 | 1.0 |
| 11.90 | 100.0 | 1.0 |
| 12.00 | 0.0 | 1.0 |
| 13.00 | 0.0 | 1.0 |

Gradient B

| | | |
|---|---|---|
| 0.00 | 3.0 | 1.0 |
| 3.00 | 30.0 | 1.0 |
| 8.25 | 33.0 | 1.0 |
| 8.30 | 33.0 | 1.5 |
| 10.30 | 34.0 | 1.5 |
| 12.00 | 50.0 | 1.5 |
| 12.10 | 100.0 | 1.5 |
| 13.50 | 100.0 | 1.5 |
| 14.00 | 3.0 | 1.0 |
| 19.00 | 3.0 | 1.0 |

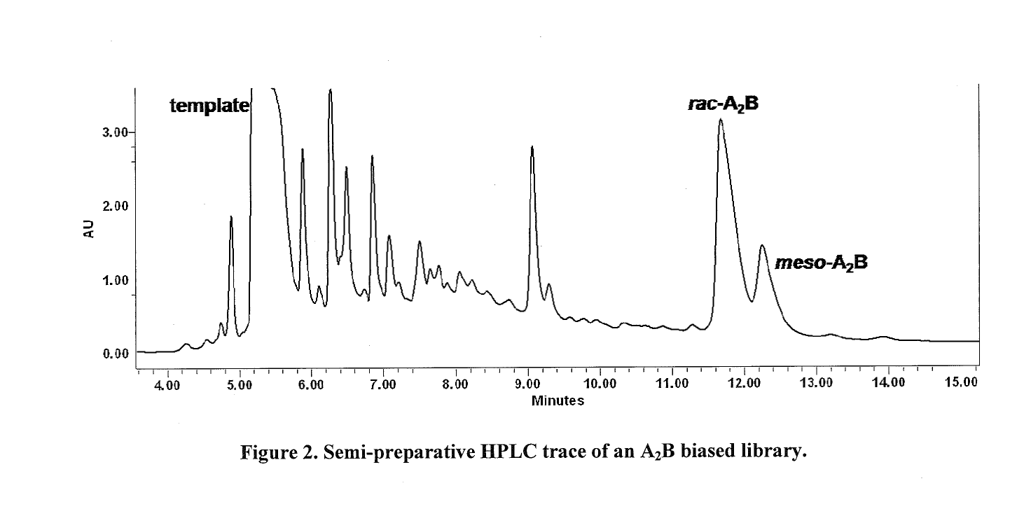

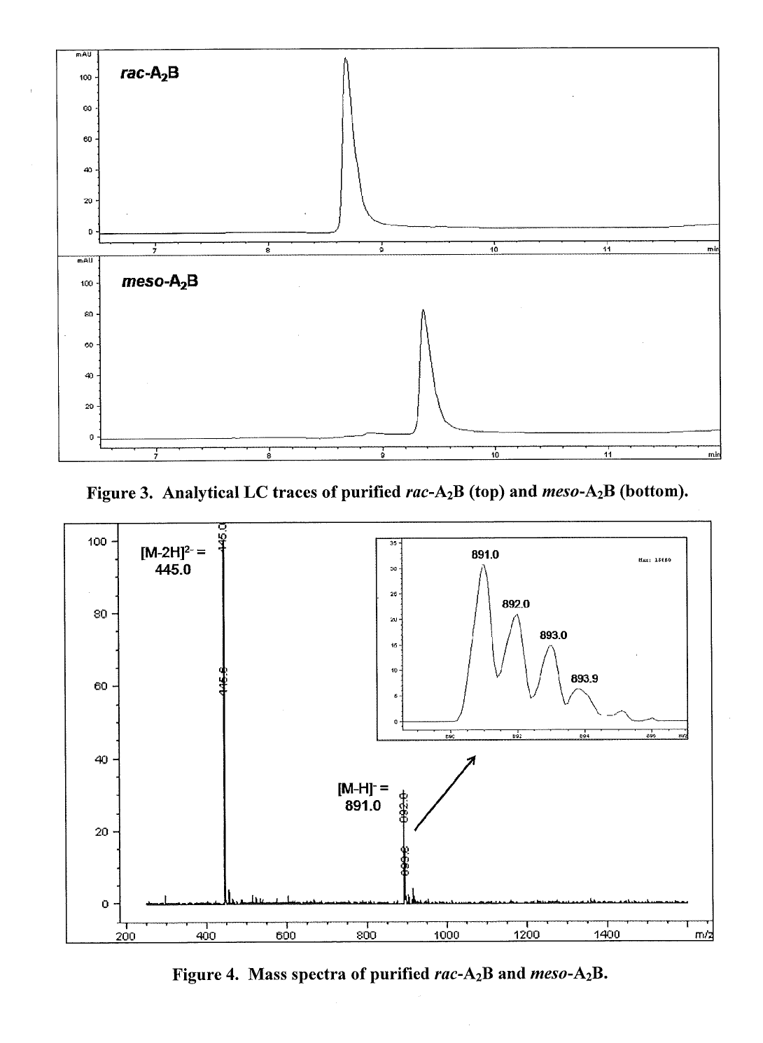

Rac-A₂B and meso-A₂B isolation. The receptors were isolated by semi-preparative HPLC using 5.0 mM A, 2.5 mM B and 7.5 mM Ac—KMe₃-G-NH₂. Upon equilibration the library was filtered, and 0.5 mL injections were chromatographed using standard peptide synthesis mobile phases A and B (0-50% B 0-5 min, then held at 50% B 5-20 min) with a flow rate of 4.0 mL/min. Optimal separation was achieved using a column heater set to 40° C. The two A₂B peaks from 11.5-12.5 minutes were collected separately (FIG. 2) and analyzed for purity by analytical LC-MS. A second and sometimes third purification was required to achieve pure meso-A₂B (FIG. 3). Both peaks were indistinguishable by mass (FIG. 4).

NMR Measurements. Trimethyl lysine peptide NMR samples were made in 50 mM pD 9.0 sodium borate buffer (referenced to DSS) in the absence and presence of excess rac-A₂B. Samples were analyzed on a Varian Inova 600 MHz instrument at 25° C. 1D NMR spectra were collected with 64 scans using a 1-1.5 second presaturation or solvent suppression.

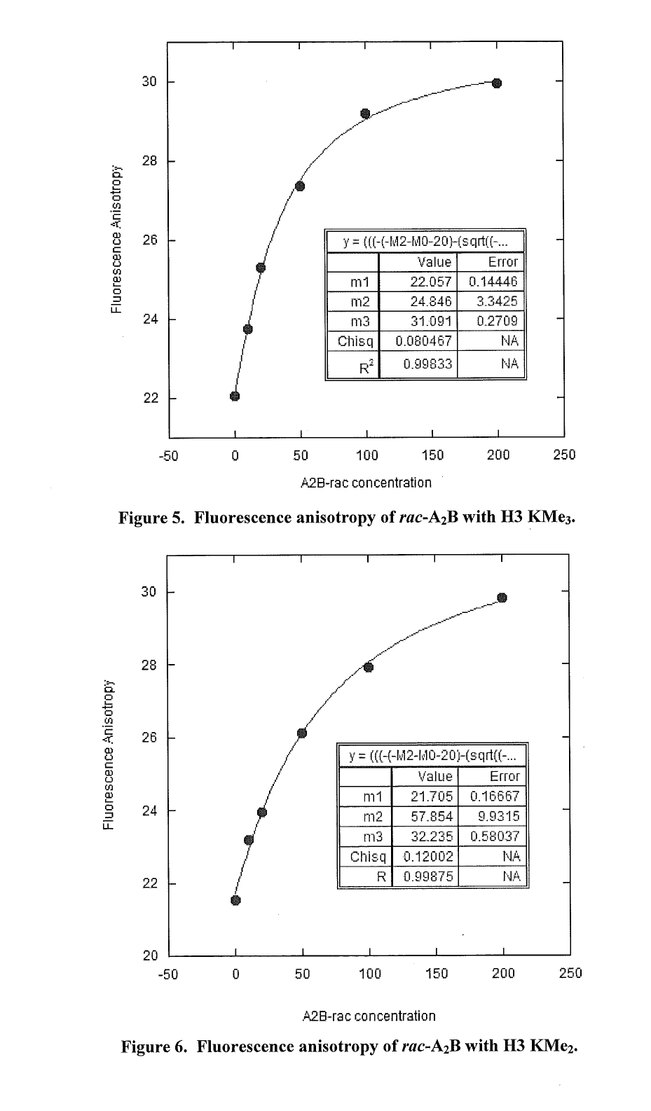

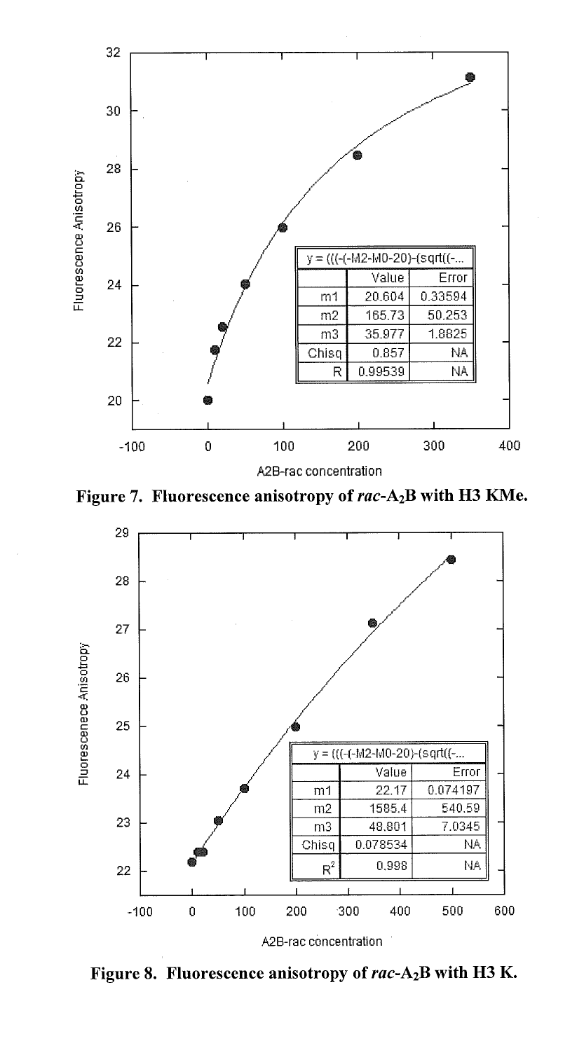

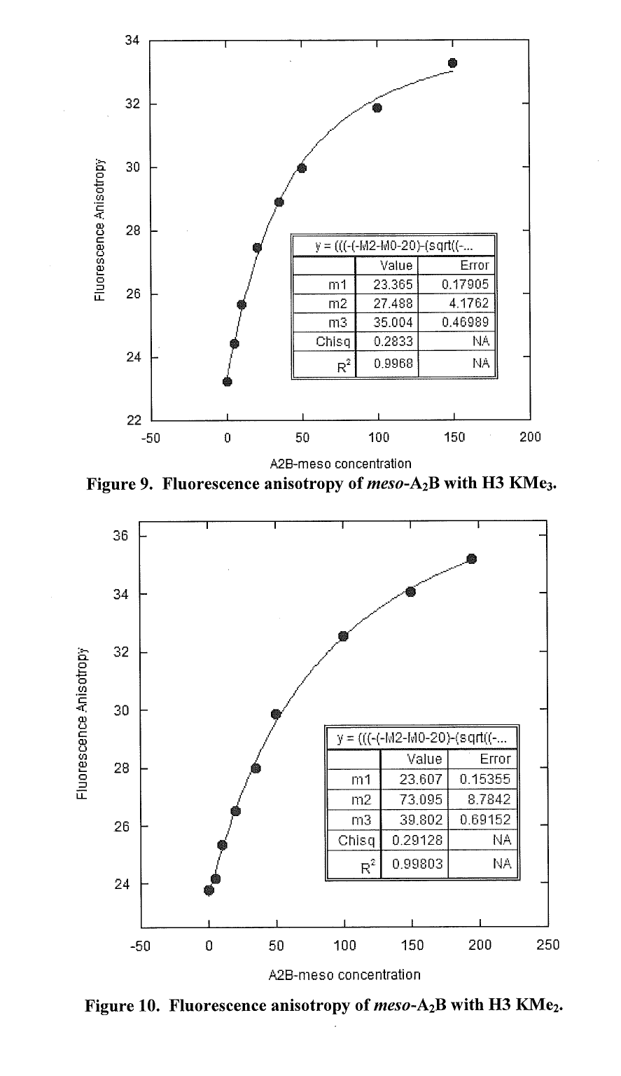

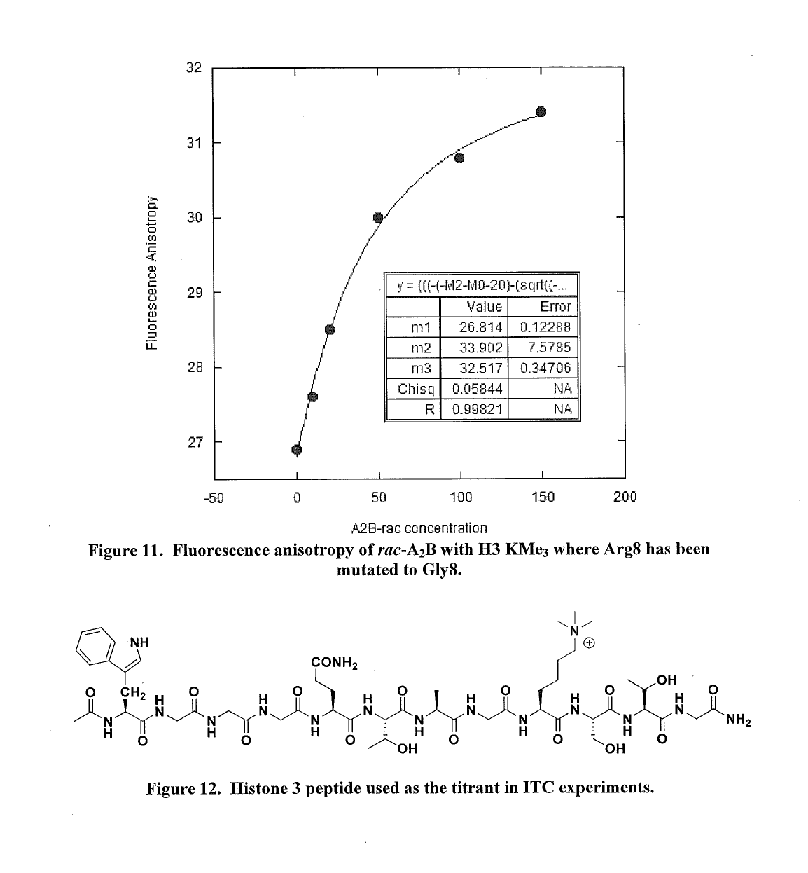

Fluorescence Anisotropy. Binding assays were performed with purified rac- or meso-A₂B and fluorescein labeled histone 3 peptides. Assays were prepared in 384-well plates (Corning) with a total volume of 50 μL per well, containing 20 μM labeled peptide and increasing concentrations of A₂B in buffer (10 mM phosphate pH 8.5). Plates were spun down and allowed to incubate for at least 30 minutes before analysis. Fluorescence anisotropy was measured on a PHERAstar (BMG Labtech) using FP485, 520A, and 520B filters at 27° C. The anisotropy data was plotted as a function A₂B concentration and each plot was fitted in KaleidaGraph to the following equation (Wang, Y.; Killian, J.; Hamasaki, K.; Rando, R. R. Biochemistry 1996, 35, 12338-12346):

$$r = ( ( \frac{( a + x + {k}_{d} ) \pm \sqrt{{( - a - x - {k}_{d} )}^{2} - 4 ( a \cdot x )}}{2 \cdot a} ) - ( {r}_{∞} - {r}_{o} ) ) + {r}_{o}$$

where r is fluorescence anisotropy, rₒ is the initial anisotropy value, r_{∞} is the maximum anisotropy value, a is the peptide concentration, x is the concentration of A₂B, and k_{d} is the dissociation constant. All measurements were taken in duplicate or triplicate. Data is given in FIGS. 5-11.

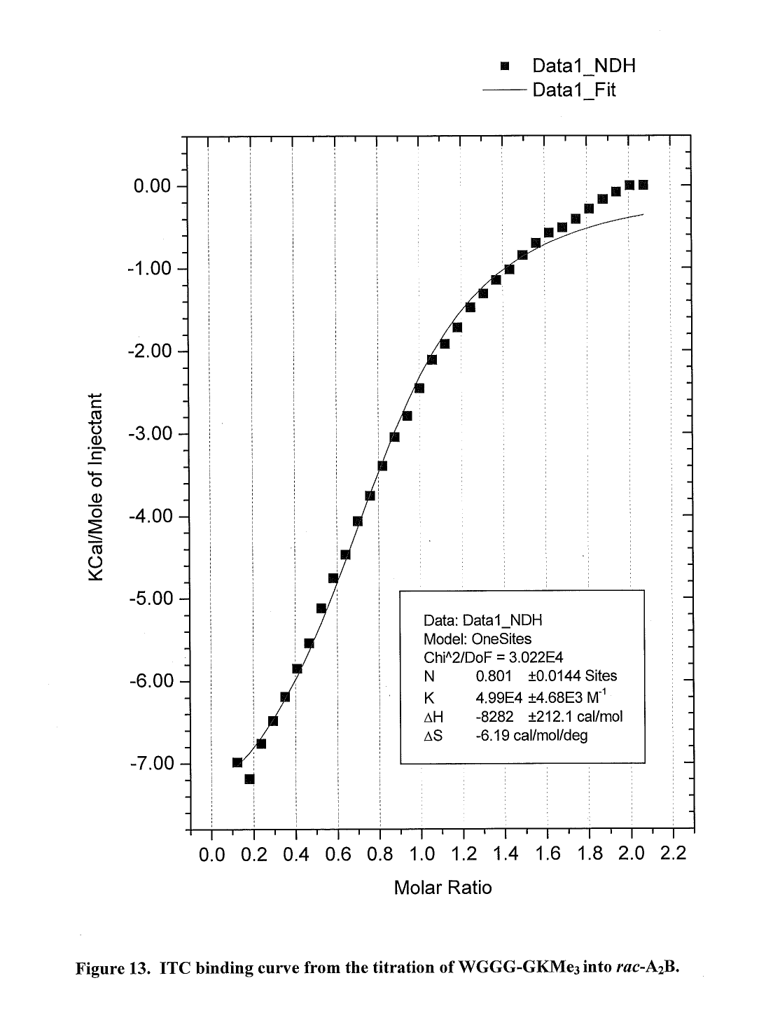

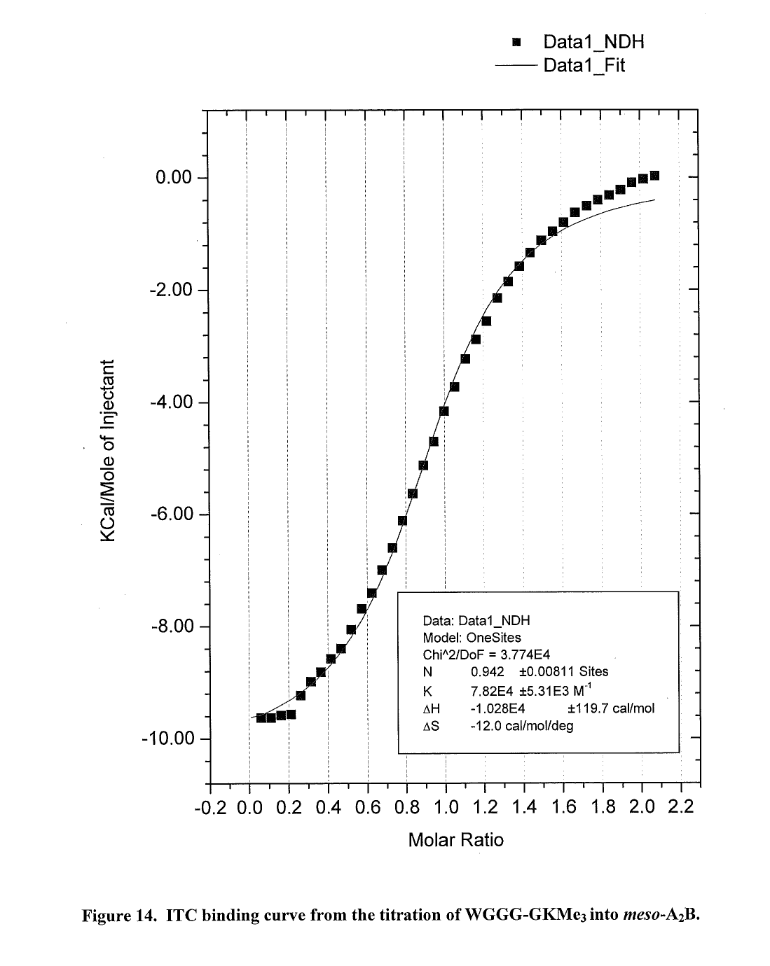

Isothermal Titration calorimetry. Isothermal titration calorimetry measurements were conducted using an ITC200 from MicroCal, LLC to verify the binding data obtained by fluorescence anisotropy. A single 0.2 μL aliquot followed by 38 aliquots of 1 μL were titrated into the calorimetric cell every 2.5 minutes. The titrant used to determine the binding to KMe₃ was a 2.0 mM solution of the H3 tail peptide in FIG. 12 (in 10 mM phosphate buffer, pH 8.5), containing a Trp for concentration determination and separated from the natural sequence by 3 glycine spacers. This peptide also contained Gly8 as opposed to Arg8 which is found in the native sequence. It has been shown that this mutation has minimal affect on the overall binding affinity. The cell was filled with a 0.2 mM solution of either rac- or meso-A₂B (in 10 mM phosphate buffer, pH 8.5). All measurements were carried out at 26° C. It was found by ITC that rac-A₂B binds KMe₃ with a K_{d} of 20.0 μM, whereas meso-A₂B binds KMe₃ with a K_{d} of 12.8 μM (FIGS. 13 and 14).

### REFERENCES

1. Bhaumik, S. R.; Smith, E.; Shilatifard, A. Nature Struct. Molec. Biol. 2007, 14, 1008-1016.
2. Wang, G. G.; Allis, C. D.; Chi, P. Trends in Molec. Med. 2007, 13, 363-372.
3. Jenuwein, T.; Allis, C. D. Science 2001, 293, 1074-1080.
4. Kouzarides, T. Cell 2007, 128, 693-705.
5. Martin, C.; Zhang, Y. Nature Rev. Molec. Cell Biol. 2005, 6, 838-849.
6. Blow, N. Nature 2007, 447, 741-744.
7. Kazanecki, C. C.; Kowalski, A. J.; Ding, T.; Rittling, S. R.; Denhardt, D. T. J. Cell. Biochem. 2007, 102, 925-935.
8. An aptamer that binds acetyl lysine was recently reported; see: Williams, B. A. R.; Lin, L.; Lindsay, S. M.; Chaput, J. C. J. Am. Chem. Soc. 2009, 131, 6330-6331.
9. For reviews, see: (a) Corbett, P. T.; Leclaire, J.; Vial, L.; West, K. R.; Wietor, J-L.; Sanders, J. K. M.; Otto, S. Chem. Rev. 2006, 106, 3652-3711. (b) Lehn, J-M. Chem. Eur. J. 1999, 5, 2455-2463. (c) Ladame, S. Org. Biomol. Chem. 2008, 6, 219-226. (d) Ludlow, R. F.; Otto, S. Chem. Soc. Rev. 2008, 37, 101-108. (e) Otto, S. Curr. Opin. Drug Disc. Devel. 2003, 6, 509-520.
10. Hughes, R. M.; Wiggins, K. R.; Khorasanizadeh, S.; Waters, M. L. Proc. Nat. Acad. Sci. USA 2007, 104, 11184-11188.
11. Jacobs, S. A.; Khorasanizadeh, S. Science 2002, 295, 2080-2083.
12. Nielsen, P. R.; Nietlispach, D.; Mott, H. R.; Callaghan, J.; Bannister, A.; Kouzarides, T.; Murzin, A. G.; Murzina, N. V.; Laue, E. D. Nature 2002, 416, 103-107.
13. Taverna, S. D.; Li, H; Ruthenburg, A. J.; Allis, C. D.; Patel, D. J. Nat. Struct. Mol. Biol. 2007, 14, 1025-1040.
14. Ma, J. C.; Dougherty, D. A. Chem. Rev. 1997, 97, 1303-1324.
15. Otto, S.; Furlan, R. L. E.; Sanders, J. K. M. Science 2002, 297, 590-593. (b) Corbett, P. T.; Sanders, J. K. M.; Otto, S. Chem. Eur. J. 2008, 14, 2153-2166.
16. Otto, S.; Furlan, R. L. E.; Sanders, J. K. M. J. Am. Chem. Soc. 2000, 122, 12063-12064.
17. Ramström, O.; Lehn, J.-M. ChemBioChem 2000, 1, 41-48.
18. No NMR studies were performed with meso-A2B because of limited availability of this compound due to significant co-elution with rac-A2B.

The foregoing is illustrative of the present invention, and is not to be construed as limiting thereof. The invention is defined by the following claims, with equivalents of the claims to be included therein.

1. A method of making a synthetic organic receptor that specifically binds to a modified amino acid, comprising:
reacting a plurality of monomers in a dynamic combinatorial library in the presence of a protein or peptide to form a reaction product, said protein or peptide comprising a modified nucleic acid, with said reacting step carried out under conditions in which at least one oligomer that specifically binds to said modified amino acid is selectively amplified in said reaction product; and then
isolating or identifying from said reaction product at least one oligomer that specifically binds to said modified amino acid.

2. The method of claim 1, wherein said modified amino acid is selected from the group consisting of monomethyl lysine, dimethyl lysine, trimethyl lysine, acetyl lysine, monomethyl arginine, symmetric dimethyl arginine, asymmetric dimethyl arginine, citrulline, phosphoserine, phosphothreonine, phosphotyrosine, 3-nitrotyrosine, oxomethionine, S-methylmethionine, S-adenosylmethionine, and glycosyl amino acids.

3. The method of claim 2, wherein said glycosyl amino acid is selected from the group consisting of glycosylated serine, glycosyl threonine, glycosyl tyrosine, and glycosyl asparigine.

4. The method of claim 1, wherein said modified amino acid is a glycosylated amino acid comprising a glycosyl selected from the group consisting of glucose, galactose, mannose, fucose, GalNAc, GIcNAc and NANA.

5. The method of claim 1, wherein each of said monomers is a compound of the formula A-B-C, wherein:
A is a first reactive group;
B is a linking group; and
C is a second reactive group that reversibly covalently bonds to said first reactive group.

6. The method of claim 5, wherein said first and second reactive groups are each independently selected from the group consisting of amino groups, aldehyde groups, keto groups, thiol groups, thioester groups, olefinic groups, alcohol groups, carbonyl groups, hydrazine groups, hydroxylamine groups and borate groups.

7. The method of claim 5, wherein each of said linking groups is independently selected from the group consisting of alkyl, aryl, alkylaryl, alkylarylalkyl, amide, oligoamide, ester, urea, guanidinium, and ether linking groups.

8. The method of claim 1, wherein said oligomer is a cyclic molecule consisting of from 2 to 20 monomers covalently coupled to one another.

9. The method of claim 8, wherein said monomers in said oligomer are coupled to one another by disulfide, imine, acyl-hydrazone, amide, acetal, ester, or thioester linkages.

10. The method of claim 9, wherein said reacting step is carried out in an aqueous media.

11. The method of claim 1, wherein said isolating or identifying step is carried out by affinity binding or chromatography.

12. A synthetic organic receptor that specifically binds to a modified amino acid, for use in detecting a protein or peptide comprising said modified amino acid;
wherein said receptor is (a) an oligomer reaction product of a dynamic combinatorial library of monomers, or (b) an analog thereof.

13. The synthetic organic receptor of claim 12, wherein said modified amino acid is selected from the group consisting of monomethyl lysine, dimethyl lysine, trimethyl lysine, acetyl lysine, monomethyl arginine, symmetric dimethyl arginine, asymmetric dimethyl arginine, citrulline, phosphoserine, phosphothreonine, phosphotyrosine, 3-nitrotyrosine, oxomethionine, S-methylmethionine, S-adenosylmethionine, and glycosyl amino acids.

14. The synthetic organic receptor of claim 13, wherein said modified amino acid is glycosyl amino acid is selected from the group consisting of glycosylated serine, glycosyl threonine, glycosyl tyrosine, and glycosyl asparigine.

15. The synthetic organic receptor of claim 13, wherein said modified amino acid is a glycosylated amino acid comprising a glycosyl selected from the group consisting of glucose, galactose, mannose, fucose, GalNAc, GIcNAc and oligosaccharides.

16. The synthetic organic receptor of claim 12, wherein said dynamic oligomer is formed from the oligomerization of monomers in a dynamic combinatorial library, and each of said monomers is a compound of the formula A-B-C, wherein:
A is a first reactive group;
B is a linking group; and
C is a second reactive group that reversibly covalently bonds to said first reactive group.

17. The synthetic organic receptor of claim 16, wherein said first and second reactive groups are each independently selected from the group consisting of amino groups, aldehyde groups, keto groups, thiol groups, thioester groups, olefinic groups, alcohol groups, carbonyl groups, hydrazine groups, hydroxylamine groups and borate groups.

18. The synthetic organic receptor of claim 16, wherein each of said linking groups is independently selected from the group consisting of alkyl, aryl, alkylaryl, and alkylarylalkyl, amide, oligoamide, ester, urea, guanidinium, and ether linking groups.

19. The synthetic organic receptor of claim 12, wherein said oligomer is a cyclic molecule consisting of from 2 to 20 monomers covalently coupled to one another.

20. The synthetic organic receptor of claim 12, wherein said monomers in said oligomer are coupled to one another by disulfide, imine, acyl-hydrazone, amide, acetal, ester, or thioester linkages.

21. The synthetic organic receptor of claim 12, wherein said receptor is coupled to a detectable group or solid support.

22. A method of collecting or detecting a protein or peptide comprising a modified amino acid, said method comprising:
contacting said protein or peptide to a synthetic organic receptor of claim 12.

23. The method of claim 22, wherein said contacting step is carried out in an aqueous media.

24. The method of claim 22, wherein said modified amino acid is selected from the group consisting of monomethyl lysine, dimethyl lysine, trimethyl lysine, acetyl lysine, monomethyl arginine, symmetric dimethyl arginine, asymmetric dimethyl arginine, citrulline, phosphoserine, phosphothreonine, phosphotyrosine, 3-nitrotyrosine, oxomethionine, S-methylmethionine, S-adenosylmethionine, and glycosyl amino acids.

25. The synthetic organic receptor of claim 24, wherein said modified amino acid is a glycosyl amino acid is selected from the group consisting of glycosylated serine, glycosyl threonine, glycosyl tyrosine, and glycosyl asparigine.

26. The synthetic organic receptor of claim 24, wherein said modified amino acid is a glycosylated amino acid comprising a glycosyl selected from the group consisting of glucose, galactose, mannose, fucose, GalNAc, GIcNAc and oligosaccharides.
